(19) United States
(12) Patent Application Publication
(10) Pub. No.: US 2009/0258385 A1
(43) Pub. Date: Oct. 15, 2009

(54) METHOD OF ANALYZING CELLS BY FLOW CYTOMETRY, USE OF AN ANTIOXIDANT AND/OR FREE RADICAL-SCAVENGING AGENT IN SUCH A METHOD AND INCUBATION BUFFER COMPRISING AN ANTIOXIDANT AND/OR FREE RADICAL-SCAVENGING AGENT

(75) Inventors: Remi Letestu, Noisy Le Grand (FR); Christine Le Roy, Paris (FR); Florence Cymbalista, Paris (FR); Nadine Varin-Blank, Sartrouville (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Hopitaux de Paris

(21) Appl. No.: 12/407,676

(22) Filed: Mar. 19, 2009

(30) Foreign Application Priority Data
Mar. 20, 2008 (FR) PCT/FR2008/000380

(51) Int. Cl. C12Q 1/04

(52) U.S. Cl. 435/34

(57) ABSTRACT

The invention relates to a method of analyzing cells by flow cytometry and the use of an antioxidant and/or free radical-scavenging agent in such a method. It also relates to an incubation buffer comprising an antioxidant and/or free radical-scavenging agent. The method of the invention comprises a step for labeling cells in a labeling solution comprising an incubation buffer and the labeling antibody or antibodies conjugated with at least one fluorochrome which is an APC tandem and an antioxidant and/or free radical-scavenging agent. The invention finds application in the field of the analysis of cells by flow cytometry, in particular.

### CROSS-REFERENCE TO RELATED APPLICATION

This application claims priority from PCT application PCT/FR2008/000380 filed Mar. 20, 2008.

### FIELD AND BACKGROUND OF THE INVENTION

The invention relates to a method of analyzing cells by flow cytometry and the use of an antioxidant and/or free radical-scavenging agent in such a method. It also relates to an incubation buffer comprising an antioxidant and/or free radical-scavenging agent.

The methodology of flow cytometry allows the simultaneous measurement of distinct morphological and fluorescent parameters at the cellular level. The development of multicolor systems has many advantages such as providing a large quantity of information at the level of the cell itself, a precise measurement even in rare cell populations, and identification of novel cell characteristics.

In particular, this technology is crucial for the functional identification of subpopulations of cells in the entire hematopoietic system, allowing better understanding of the physiology of the cell and of the physiopathology of malignant pathologies.

It is therefore regularly used in hematology for the diagnosis or therapeutic monitoring of various conditions where it allows the detection of pathological blood cells and their differentiation relative to healthy blood cells.

As flow cytometric experiments are becoming increasingly complex, the demand for novel fluorochromes with different properties is increasing.

Fluorochromes (fluorescent dyes) absorb light at given wavelengths and in turn emit their fluorescence energy at a higher wavelength.

Among the novel fluorochromes, the tandem APC-Cy7 (allophycocyanin-cyanine 7) and its analog APC-H7 (allophycocyanin-Hilite®7-BD) are fluorochromes called APC tandems exhibiting similar spectral properties with an absorption maximum at approximately 650 nm. Under excitation by a red laser (HeNe, 633 nm), the APC fluorochrome present in the APC tandem transfers its energy to the cyanine dye, which in turn emits at 767 nm. The APC tandem fluorochromes are conjugated with antibodies directed against cellular antigens.

However, the excitation of these antibody-APC tandem conjugates can result in emission in the APC channel. This emission, which is commonly observed by flow cytometrists, leads to false-positive events and to reduced labeling sensitivity.

Up until now, emission in the APC channel was explained by at least two mechanisms. First of all, a nonoptimum association of the APC dye and the Cy7 or H7 dyes can lead to emission of fluorescence of APC, reflecting incomplete reabsorption by the Cy7 or H7 dyes of the fluorescence emitted by the APC molecule. Secondly, a lack of stability of the APC-Cy7 and APC-H7 tandems can result in an “unwanted” APC signal. This mechanism is generally called “decoupling phenomenon”.

The lack of stability of APC tandems (that is to say tandems of dyes of which one is APC) is generally attributed to photodegradation. Indeed, it is known that fluorochromes are sensitive to light. That is why the analyses, including the preparation of the cells for analysis (lysis, immunolabeling and the like) are carried out with protection from light, which is a precaution that is particularly advanced for conjugates comprising APC tandems.

Besides, ascorbic acid is an antioxidant capable of countering the harmful effect of oxidants and free radicals.

The L form of ascorbic acid, vitamin C, furthermore has vitamin activity.

However, vitamin C is very fragile in solution, it is destroyed upon contact with air, by light or heat.

The aim of the invention is to provide measures which make it possible to carry out analyses of cells by flow cytometry using, as fluorochromes, the APC tandems, without the phenomenon of decoupling occurring or such that the latter is at least inhibited.

### SUMMARY OF THE INVENTION

To this end, the invention proposes a method for analyzing cells by flow cytometry comprising a step of labeling the cells by incubation in a labeling solution comprising an incubation buffer and labeling antibodies conjugated with at least one fluorochrome which is an APC tandem, characterized in that at least one antioxidant and/or free radical-scavenging agent is introduced into the labeling solution.

The expression APC tandem is understood to mean an APC-Cy7 tandem, an APC-H7 tandem or an APC-Alexa 750 tandem.

Preferably, the cells are blood cells.

Preferably still, the antioxidant and/or free radical-scavenging agent is ascorbic acid.

More preferably, the antioxidant and/or free radical-scavenging agent is vitamin C.

When the antioxidant and/or free radical-scavenging agent is vitamin C, and when the cells are lymphocytes, the vitamin C is present in the solution analyzed by flow cytometry at a final concentration of between 0.1 mM and 10 mM inclusive, preferably at a final concentration of between 1 mM and 10 mM inclusive, most preferably at a concentration of 1 mM.

When the antioxidant and/or free radical-scavenging agent is vitamin C and the cells are monocytes, the vitamin C is present in the solution analyzed by flow cytometry at a final concentration of between 1 mM and 10 mM inclusive, most preferably at a concentration of 1 mM.

When the antioxidant and/or free radical-scavenging agent is vitamin C and the cells analyzed are neutrophilic polynuclear cells, the vitamin C is present at a final concentration in the analyzed solution of between 0.25 mM and 10 mM inclusive, preferably between 0.75 mM and 4 mM inclusive, more preferably between 0.75 mM and 4 mM inclusive, most preferably of 0.75 mM.

The invention also proposes the use of an antioxidant and/or free radical-scavenging agent for inhibiting the degradation of the tandem fluorochromes APC-Cy7, APC-H7 or APC-Alexa 750 during flow cytometry analysis of cells.

Preferably, in the use of the invention, the cells are blood cells.

Also preferably, in the use of the invention, the antioxidant and/or free radical-scavenging agent is ascorbic acid, most preferably in the L form (vitamin C).

When the antioxidant and/or free radical-scavenging agent is vitamin C, and when the cells are lymphocytes, the vitamin C is present in the solution analyzed by flow cytometry at a final concentration of between 0.1 mM and 10 mM inclusive, preferably at a final concentration of between 1 mM and 10 mM inclusive, most preferably at a concentration of 1 mM.

When the antioxidant and/or free radical-scavenging agent is vitamin C and the cells are monocytes, the vitamin C is present in the solution analyzed by flow cytometry at a final concentration of between 1 mM and 10 mM inclusive, most preferably at a concentration of 1 mM.

When the antioxidant and/or free radical-scavenging agent is vitamin C and the cells analyzed are neutrophilic polynuclear cells, the vitamin C is present at a final concentration in the analyzed solution of between 0.25 mM and 10 mM inclusive, preferably between 0.75 mM and 4 mM inclusive, more preferably between 0.75 mM and 4 mM inclusive, most preferably of 0.75 mM.

The invention also proposes an incubation buffer comprising an antioxidant and/or free radical-scavenging agent.

Preferably, in the incubation buffer of the invention, the antioxidant and/or free radical-scavenging agent is ascorbic acid, more preferably in the L form, vitamin C.

### BRIEF DESCRIPTION OF THE DRAWINGS

The invention will be better understood and other features and advantages thereof will emerge more clearly on reading the explanatory description which follows and which refers to the figures in which:

FIG. 1 is an image obtained from the cytometer showing the intensity of fluorescence, in arbitrary units, (x-axis) of neutrophilic polynuclear cells, of monocytes and of lymphocytes as a function of their granularity, in arbitrary units, labeled with anti-CD45 (α-CD45) antibodies conjugated with the APC-Cy7 tandem,

FIG. 2 shows the intensity of the fluorescence signal of lymphocytes labeled with anti-CD45 antibodies conjugated with the APC-Cy7 tandem in the APC channel and in the APC-Cy7 channel just after incubation (T0),

FIG. 3 shows the intensity of the fluorescence signal of lymphocytes labeled with anti-CD45 antibodies conjugated with the APC-Cy7 tandem in the APC channel and in the APC-Cy7 channel 30 minutes after incubation (T30),

FIG. 4 shows the variation of the percentage of decoupling observed at time T0 and T30 of lymphocytes labeled with anti-CD45 antibodies conjugated with the APC-Cy7 tandem,

FIG. 5 is an image obtained from the cytometer showing the intensity of fluorescence, in arbitrary units, (x-axis) of neutrophilic polynuclear cells, of monocytes and of lymphocytes as a function of their granularity, in arbitrary units, labeled with anti-CD45 (α-CD45) antibodies conjugated with the APC-H7 tandem,

FIG. 6 shows the intensity of the fluorescence signal of lymphocytes labeled with anti-CD45 antibodies conjugated with the APC-H7 tandem in the APC channel and in the APC-H7 channel just after incubation (T0),

FIG. 7 shows the intensity of the fluorescence signal of lymphocytes labeled with anti-CD45 antibodies conjugated with the APC-H7 tandem in the APC channel and in the APC-H7 channel 30 minutes after incubation (T30),

FIG. 8 shows the variation of the percentage of decoupling observed at time T0 and T30 of lymphocytes labeled with anti-CD45 antibodies conjugated with the APC-H7 tandem,

FIG. 9 is a schematic representation of the mode of binding of the antibodies conjugated with the APC tandems to beads Compbeads® from Becton Dickinson and to cells,

FIG. 10 shows the variation of the percentage of decoupling, as a function of time, observed during flow cytometry analysis of beads Compbeads® from Becton Dickinson labeled with an anti-CD45 antibody conjugated with the APC-Cy7 tandem and of lymphocytes labeled with an anti-CD45 antibody conjugated with the APC-Cy7 tandem,

FIG. 11 shows the variation of the percentage of decoupling, as a function of time, observed during flow cytometry analysis of beads (beads Compbeads® from Becton Dickinson) labeled with an anti-CD45 antibody conjugated with the APC-H7 tandem and of lymphocytes labeled with an anti-CD45 antibody conjugated with the APC-H7 tandem,

FIG. 12 shows the variation of the percentage of decoupling, as a function of time, observed during flow cytometry analysis of lymphocytes labeled with an anti-CD20 (α-CD20) antibody conjugated with the APC-Cy7 tandem, and of lymphocytes labeled with an anti-CD20 antibody conjugated with the APC-H7 tandem,

FIG. 13 shows the variation of the percentage of decoupling, as a function of time, observed during flow cytometry analysis of lymphocytes labeled with an anti-CD3 (α-CD3) antibody conjugated with the APC-Cy7 tandem, and of lymphocytes labeled with an anti-CD3 antibody conjugated with the APC-H7 tandem,

FIG. 14 shows the variation of the percentage of decoupling, as a function of time, observed during flow cytometry analysis of lymphocytes labeled with an anti-CD45 antibody conjugated with the APC-Cy7 tandem or with the APC-H7 tandem, and of neutrophilic polynuclear cells labeled with an anti-CD45 antibody conjugated with the APC-Cy7 tandem or with the APC-H7 tandem,

FIG. 15 shows the variation of the percentage of decoupling, as a function of time, observed during flow cytometry analysis of monocytes labeled with an anti-CD45 antibody conjugated with the APC-Cy7 tandem or with the APC-H7 tandem,

FIG. 16 shows the variation of the percentage of decoupling, as a function of time, observed during flow cytometry analysis of:

a) lymphocytes labeled with an anti-CD45 antibody conjugated with the APC-Cy7 tandem, with protection from light,

b) lymphocytes labeled with an anti-CD45 antibody conjugated with the APC-Cy7 tandem, with exposure to light,

c) monocytes labeled with an anti-CD45 antibody conjugated with the APC-Cy7 tandem, with protection from light,

d) monocytes labeled with an anti-CD45 antibody conjugated with the APC-Cy7 tandem, with exposure to light,

e) neutrophilic polynuclear cells labeled with an anti-CD45 antibody conjugated with the APC-Cy7 tandem, with protection from light,

f) neutrophilic polynuclear cells labeled with an anti-CD45 antibody conjugated with the APC-Cy7 tandem, with exposure to light,

FIG. 17 shows the variation of the percentage of decoupling, as a function of time, observed during flow cytometry analysis of:

a) lymphocytes labeled with an anti-CD45 antibody conjugated with the APC-H7 tandem, with protection from light,

b) lymphocytes labeled with an anti-CD45 antibody conjugated with the APC-H7 tandem, with exposure to light,

c) monocytes labeled with an anti-CD45 antibody conjugated with the APC-H7 tandem, with protection from light,

d) monocytes labeled with an anti-CD45 antibody conjugated with the APC-H7 tandem, with exposure to light,

e) neutrophilic polynuclear cells labeled with an anti-CD45 antibody conjugated with the APC-H7 tandem, with protection from light,

f) neutrophilic polynuclear cells labeled with an anti-CD45 antibody conjugated with the APC-H7 tandem, with exposure to light,

FIG. 18 shows the variation in the mean intensity of fluorescence, as a function of time, of beads (beads Compbeads® from Becton Dickinson) labeled with an anti-CD45 antibody conjugated with the APC-Cy7 tandem in the presence and absence of light,

FIG. 19 shows the variation in the mean intensity of fluorescence, as a function of time, of beads (beads Compbeads® from Becton Dickinson) labeled with an anti-CD45 antibody conjugated with the APC-H7 tandem in the presence and absence of light,

FIG. 20 shows the variation in the mean intensity of fluorescence, as a function of time, of beads (beads Compbeads® from Becton Dickinson) labeled with an anti-CD45 antibody conjugated with the APC fluorochrome in the presence and in the absence of light,

FIG. 21 shows the variation of the percentage of decoupling, as a function of time, observed during flow cytometry analysis of:

a) unbound lymphocytes labeled with an anti-CD45 antibody conjugated with the APC-Cy7 tandem,

b) bound lymphocytes labeled with an anti-CD45 antibody conjugated with the APC-Cy7 tandem,

c) unbound neutrophilic polynuclear cells labeled with an anti-CD45 antibody conjugated with the APC-Cy7 tandem,

d) bound neutrophilic polynuclear cells labeled with an anti-CD45 antibody conjugated with the APC-Cy7 tandem,

FIG. 22 shows the variation of the percentage of decoupling, as a function of time, observed during flow cytometry analysis of:

a) lymphocytes labeled with an anti-CD45 antibody conjugated with the APC-Cy7 tandem, incubated at room temperature,

b) lymphocytes labeled with an anti-CD45 antibody conjugated with the APC-Cy7 tandem, incubated at 4° C.,

c) neutrophilic polynuclear cells labeled with an anti-CD45 antibody conjugated with the APC-Cy7 tandem, incubated at room temperature,

d) neutrophilic polynuclear cells labeled with an anti-CD45 antibody conjugated with the APC-Cy7 tandem, incubated at 4° C.,

FIG. 23 shows the variation of the percentage of decoupling, as a function of time, observed during flow cytometry analysis of unbound monocytes labeled with an anti-CD45 antibody conjugated with the APC-Cy7 tandem, and of bound monocytes labeled with an anti-CD45 antibody conjugated with the APC-Cy7 tandem,

FIG. 24 shows the variation of the percentage of decoupling, as a function of time, observed during flow cytometry analysis of monocytes labeled with an anti-CD45 antibody conjugated with the APC-Cy7 tandem, incubated at room temperature, and of monocytes labeled with an anti-CD45 antibody conjugated with the APC-Cy7 tandem, incubated at 4° C.,

FIG. 25 shows the variation of the percentage of decoupling, as a function of time, observed during flow cytometry analysis of:

a) lymphocytes labeled with an anti-CD45 antibody conjugated with the APC-Cy7 tandem, incubated without vitamin C,

b) lymphocytes labeled with an anti-CD45 antibody conjugated with the APC-Cy7 tandem, incubated in the presence of 1 mM vitamin C,

c) neutrophilic polynuclear cells labeled with an anti-CD45 antibody conjugated with the APC-Cy7 tandem, incubated without vitamin C,

d) neutrophilic polynuclear cells labeled with an anti-CD45 antibody conjugated with the APC-Cy7 tandem, incubated in the presence of 1 mM vitamin C, and

FIG. 26 shows the variation of the percentage of decoupling, as a function of time, observed during flow cytometry analysis of monocytes labeled with an anti-CD45 antibody conjugated with the APC-Cy7 tandem, incubated without vitamin C, and of monocytes labeled with an anti-CD45 antibody conjugated with the APC-Cy7 tandem, incubated in the presence of 1 mM vitamin C.

### MORE DETAILED DESCRIPTION

The terms “percentage of decoupling”, “% decoupling”, “% decoupling APC vs APC-Cy7”, “% decoupling APC vs APC-H7” are used in the preceding text and in the text which follows and in the accompanying figures to denote the ratio:

[Number of cells which, although having been labeled with an APC tandem, emit fluorescence into the APC channel (and not into the channel of the APC tandem used)]/[total number of cells which have been labeled with the APC tandem, regardless of the channel into which they emit],

this ratio being multiplied by 100.

The term “room temperature” is used in the preceding text and in the text which follows and in the accompanying figures to denote a temperature between 18° C. and 25° C. inclusive.

The invention is based on the elucidation, by the inventors, of the mechanism of impairment of the markers conjugated with the APC tandems on immunolabeled cells, which leads to the decoupling phenomenon observed during the analysis of these cells by flow cytometry using the APC tandems as fluorochromes.

To elucidate the mechanism leading to the decoupling phenomenon, the inventors analyzed, by flow cytometry, samples of cells labeled with various antibodies conjugated with the APC fluorochrome or with the APC-Cy7, APC-H7 or APC-Alexa 750 tandem fluorochromes.

The materials and methods used are the following:

Materials:

The anti-CD45, anti-CD20, anti-CD3 and anti-CD19 antibodies conjugated with the APC-Cy7 fluorochrome or with the APC-H7 fluorochrome and the lysis buffer BD Phosflow Lyse/Fix Buffer 5× were bought from BD Bioscience.

The lysis buffer was an ammonium chloride lysis buffer.

The phosphate-buffered saline (PBS) was obtained from Biomérieux, the fetal bovine serum (FBS) was bought from Pan Biotech Gmbh and heated for 30 minutes at 56° C. before use. The L-ascorbic acid (vitamin C) was obtained from SIGMA-ALDRICH.

Method:

Aliquots of human peripheral blood (containing 500,000 leukocytes) were incubated in the lysis buffer and then washed three times with 1×PBS supplemented with 2.5% FBS. The cells (500,000) were then immuno-labeled with 5 μl of each of the desired antibodies conjugated either with the desired APC tandem or with APC alone in 100 μl of incubation buffer for 20 min at room temperature, and washed three times with the PBS/FBS solution and resuspended in 200 μl of PBS/FBS solution.

The dead cells were labeled for their exclusion during analysis of the data acquired by flow cytometry using the intercalating agent 7-aminoactinomycin-D (7-AAD) from SIGMA-ALDRICH.

For each immunolabeling experiment, the cells were mixed and subjected to flow cytometry analysis on a Becton Dickinson FACSCanto II apparatus. From 10,000 to 50,000 events were acquired per test. The singlet cells were selected by a commonly used cell sorting procedure: intersection of the three windows (P1, P2 and P3) of the biparametric histograms.

For each type of cells, the percentage of decoupling was calculated as previously described using the BD FACSDIVA software.

The accompanying figures show an experiment representative of three to four repeat experiments.

First of all, the inventors verified that the degradation of the APC-Cy7 and APC-H7 tandem fluorochromes conjugated with antibodies directed against cell surface proteins occurred in the cellular model described above while causing the appearance of a signal in the APC channel.

For that, human peripheral blood cells were labeled with anti-CD45 antibodies conjugated either with the APC-Cy7 tandem or with the APC-H7 tandem.

The cells were analyzed by flow cytometry immediately after the preparation of the solution containing the cells for analysis by flow cytometry, that is to say at time T0, and 30 minutes later, that is to say at time T30.

FIG. 1 and FIG. 5 show that, as expected, the pan-leukocyte anti-CD45 antibodies conjugated with the APC tandem fluorochromes label the lymphocytes, the monocytes and the neutrophilic polynuclear leukocytes.

In these two figures, the term “PNN” means “neutrophilic polynuclear leukocyte or leukocyte”.

The lymphocyte population, labeled with an anti-CD45 antibody conjugated either with the APC-Cy7 tandem or with the APC-H7 tandem, was selected and two-dimensional plots representing the intensity of their fluorescence observed in the APC channel and in the APC-Cy7 channel, at the time T0 and at the time T30, were generated.

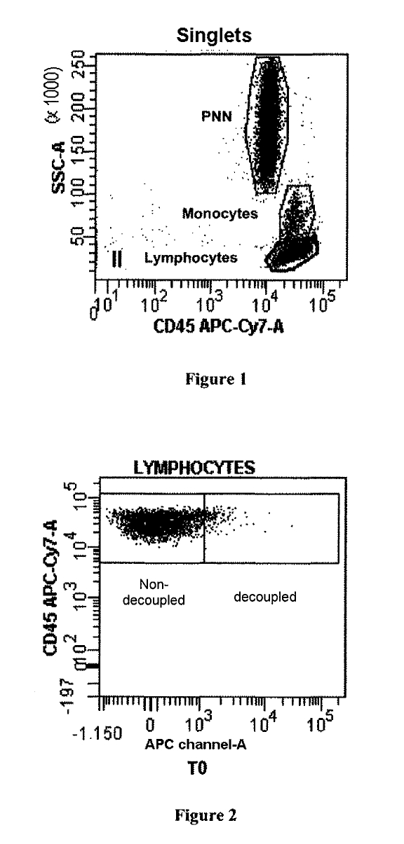

These plots are shown in FIGS. 2, 3, 5 and 7.

It can be seen from FIGS. 2 and 5 that, at the time T0, the lymphocytes appear in the form of a uniform cell population which is highly positive in the APC-Cy7 or APC-H7 channel and weakly positive in the APC channel.

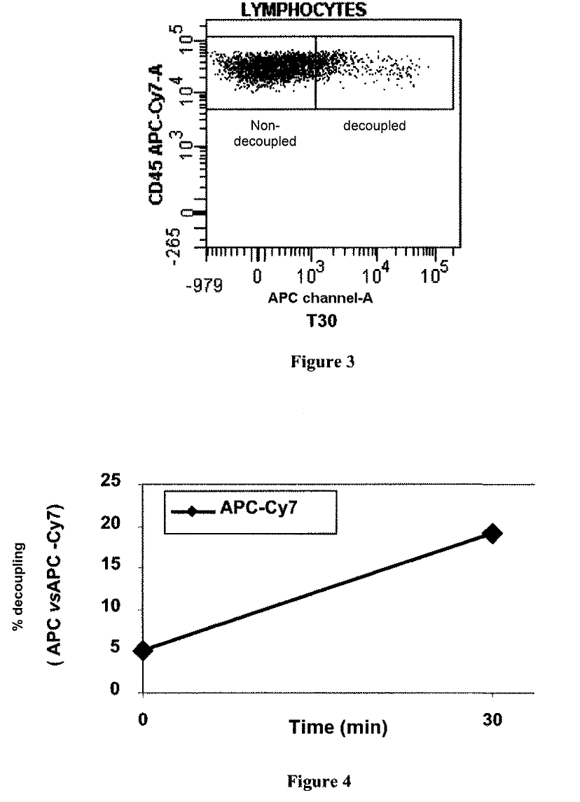

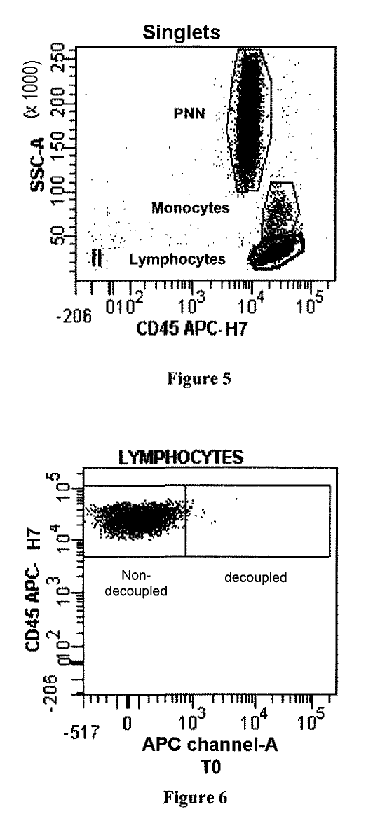

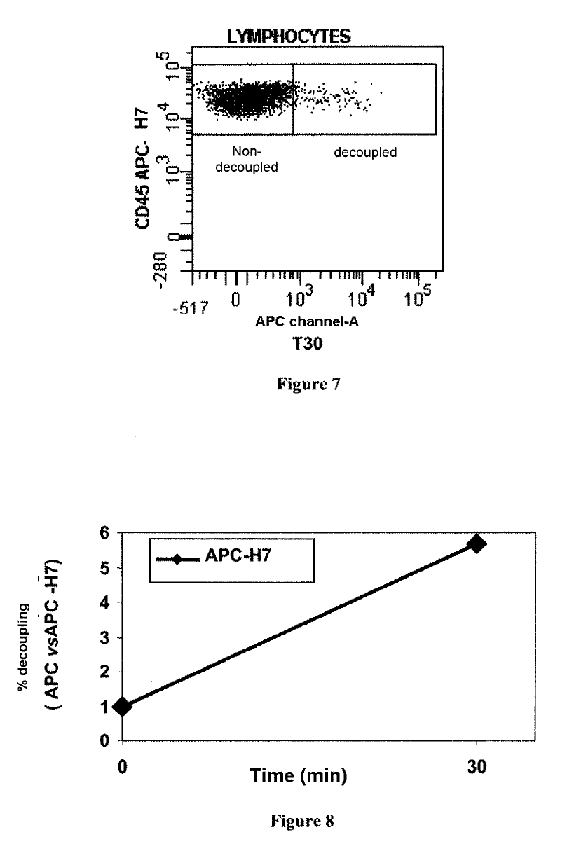

By contrast, it can be seen from FIGS. 3 and 7, that after an incubation of 30 minutes at room temperature and in the dark, the cell population of lymphocytes spreads into the APC channel.

On considering the median fluorescence intensities (MFI), an increase is observed in the APC signal at the time T30 compared with the initial time T0 among the CD45-positive cells conjugated with the APC-Cy7 tandem or with the APC-H7 tandem.

Together, these observations suggest that the increased signal in the APC channel is linked to an impairment of the APC tandem fluorochromes.

To evaluate this phenomenon, the inventors targeted a subpopulation emitting into the APC channel, that is to say positive for APC, in the population of lymphocytes positive for the APC-Cy7 or APC-H7 tandem, that is to say emitting into the APC-Cy7 or APC-H7 channel, that is to say the populations labeled “decoupled” in FIGS. 3 and 7.

The positivity threshold was determined in the APC channel at the time T0 and related to the time T30. The percentage of “decoupled” cells was expressed as the ratio of the total number of both APC- and APC tandem-positive cells to the total APC tandem-positive lymphocytes.

The results are represented in FIGS. 4 and 8.

As can be seen in these figures, the percentage decoupling of the fluorochromes APC-Cy7 and APC-H7 is higher at the time T30 than at the time T0.

Similar results were obtained with anti-CD45 antibodies coupled with the APC-Alexa 750 tandem.

The base percentage of decoupling (decoupling at the time T0) is higher for the APC-Cy7 tandem fluorochrome than for the APC-H7 tandem fluorochrome but the decoupling ratios of the two APC tandem fluorochromes have a similar variation during the incubation time, that is to say a four- to five-fold increase, as can be seen in FIGS. 4 and 8.

Together, these data suggest that the APC tandem fluorochromes conjugated with anti-CD45 antibodies are subjected to degradation over time in lymphocytes.

This first part of this study made it possible to verify that, under the experimental conditions used, the decoupling phenomenon observed during a flow cytometry analysis using the APC tandems, that is to say the APC-Cy7, APC-H7 or APC-Alexa 750 tandems, is indeed produced. This validates the model which is used.

Next, the inventors studied the influence of the cellular context on this decoupling phenomenon.

For that, they analyzed the stability of the APC tandem fluorochromes attached to lymphocytes and compared it to the stability of these same APC tandems attached to beads. This comparison makes it possible to determine whether or not it is the APC tandem which is degraded, regardless of the context in which it is used, or if the APC tandem is only degraded in the presence of cells.

The beads used in these experiments were beads Compbeads® from Becton Dickinson.

The lymphocytes, like the beads, were labeled with anti-CD45 antibodies conjugated with the APC-Cy7 tandem fluorochrome or with the APC-H7 tandem fluorochrome.

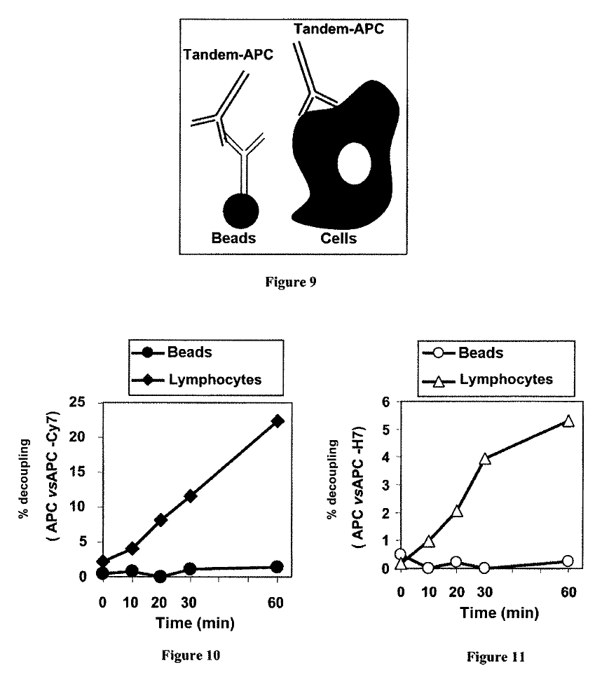

FIGS. 9 to 15 represent the data acquired during these experiments.

More specifically, FIG. 9 shows, in schematic form, the binding of the antibodies either to the beads (beads Compbeads® from Becton Dickinson) or to the lymphocytes.

As can be seen in FIG. 9, the antibodies are attached to the cellular markers by their antigen binding site while the beads are attached to the markers via the recognition of the light chains.

The variation of the percentage of decoupling observed as a function of the time when the APC-Cy7 tandem is attached either to the beads (beads Compbeads® from Becton Dickinson) or to the lymphocytes is represented in FIG. 10, and the variation of the percentage of decoupling observed as a function of the time when the beads (beads Compbeads® from Becton Dickinson) or the lymphocytes are attached to the APC-H7 tandem are represented in FIG. 11.

It can be seen from these figures that, very interestingly, the percentage of decoupling of the APC-Cy7 and APC-H7 fluorochromes increases over time for the antibodies attached to the cells alone. Indeed, no decoupling is observed for the antibodies attached to the beads.

Similar results were obtained with other antibodies such as the anti-CD20 antibody.

These data suggest that the impairment of the APC tandem fluorochromes is linked to the labeling of the cells rather than to an intrinsic instability of the fluorochromes under the conventional conditions of use of these fluorochromes.

To verify that the degradation of the APC tandem fluorochromes occurred regardless of the antibody used, several antibodies directed against lymphocyte membrane markers and conjugated either with the APC-Cy7 tandem fluorochrome or with the APC-H7 tandem fluorochrome were tested.

For that, the APC-Cy7 and APC-H7 tandem fluorochromes conjugated with antibodies directed against the anti-CD20 or anti-CD19, B lymphocytes, or the anti-CD3, T lymphocytes were used.

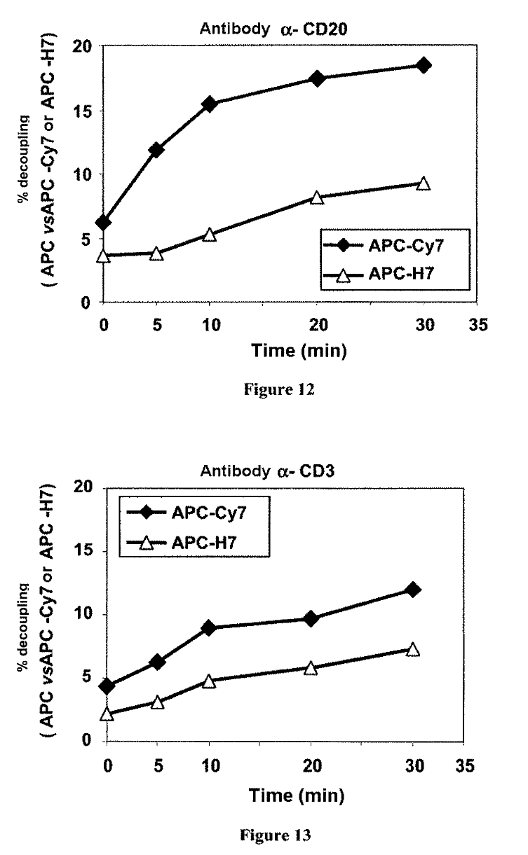

FIG. 12 shows the variation of the percentage of decoupling as a function of the incubation time, observed during the use of the anti-CD20 antibodies conjugated either with the APC-Cy7 tandem fluorochrome or with the APC-H7 tandem fluorochrome. FIG. 13 shows the variation of the percentage of decoupling, as a function of the incubation time, during the use of anti-CD3 antibodies conjugated with the APC-Cy7 tandem or APC-H7 tandem fluorochrome.

As can be seen in FIGS. 12 and 13, an increasingly high decoupling phenomenon is observed as a function of the incubation time.

Once again, the levels of degradation of the APC-Cy7 tandem fluorochrome are higher than those of the APC-H7 tandem fluorochrome.

These results also confirm that the impairment of the APC tandem fluorochromes occurs during targeting of the surface markers of both the T lymphocytes and the B lymphocytes.

The influence of the type of cells was also studied.

For that, populations of different types of leukocyte cells were labeled with pan-leukocyte anti-CD45 antibodies conjugated either with the APC-Cy7 tandem fluorochrome or with the APC-H7 tandem fluorochrome and the decoupling in each of the sub-populations was determined over time.

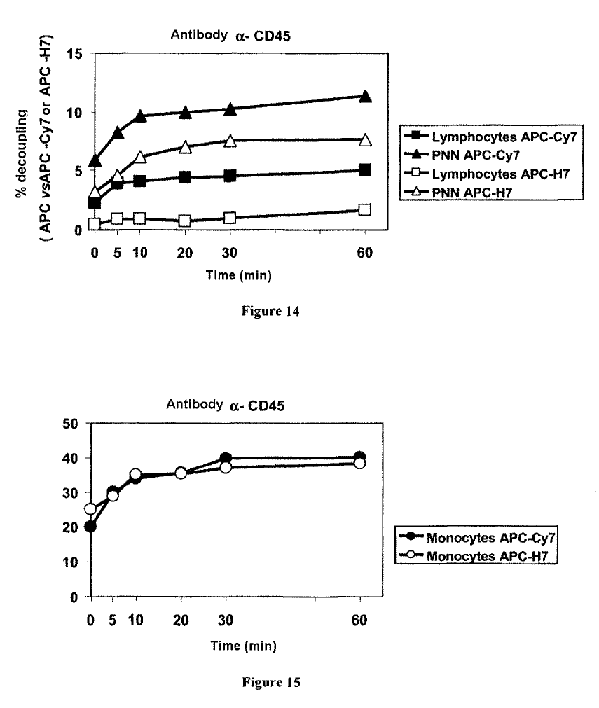

The results obtained are represented in FIG. 14 which shows the percentage decoupling as a function of time:
of lymphocytes labeled with anti-CD45 antibodies conjugated with the APC-Cy7 tandem,
of neutrophilic polynuclear cells labeled with anti-CD45 antibodies conjugated with the APC-Cy7 tandem fluorochrome,
of lymphocytes labeled with anti-CD45 antibodies conjugated with the APC-Cy7 tandem fluorochrome,
of lymphocytes labeled with anti-CD45 antibodies conjugated with the APC-H7 tandem fluorochrome, and
of neutrophilic polynuclear cells labeled with anti-CD45 antibodies conjugated with the APC-H7 tandem, and

in FIG. 15 which shows the percentage decoupling observed on monocytes labeled with anti-CD45 antibodies conjugated either with the APC-Cy7 fluorochrome or with the APC-H7 fluorochrome.

As can be seen in these figures, the percentage decoupling of the APC-Cy7 and APC-H7 tandem fluorochromes increases when the anti-CD45 antibodies are attached to lymphocytes, monocytes and granulocytes.

However, for all the conjugated APC tandems, the rate of degradation increases gradually between the lymphocytes, the monocytes and the granulocytes.

These results show that the degradation of the APC tandem fluorochromes depends on the cell type but occurs in all cases.

As a first conclusion, the above experiments of the inventors show that the decoupling phenomenon only appears in the presence of cells, regardless of this cell type, and that a decoupling phenomenon exists to a greater or lesser degree according to the cell type.

On the basis of these results, the inventors then sought the possible routes for blocking the cell-dependent degradation of the APC tandem fluorochromes.

For that, first of all, cells labeled with anti-CD45 antibodies coupled with the APC tandem fluorochromes were fixed using a buffer BD Phosflow Lyse/Fix Buffer before measuring the degradation of the fluorochrome over time.

As can be seen in FIG. 21, which represents the variation of the percentage of decoupling over time which is produced with bound or unbound lymphocytes or neutrophilic polynuclear cells labeled with anti-CD45 antibodies coupled with the APC-Cy7 tandem fluorochrome, the percentage of decoupling which is produced remains stable over time and close to the base line, that is to say to the value obtained at the time T0, in the lymphocytes and neutrophilic polynuclear leukocytes. The same is true in the monocytes, as can be seen in FIG. 23 which represents the variation of the percentage of decoupling which is produced for monocytes attached to anti-CD45 antibodies conjugated with the APC-Cy7 tandem fluorochrome, as a function of time.

The same results were obtained on these same types of cell using anti-CD45 antibodies, this time coupled with the APC-H7 tandem fluorochrome.

The strong inhibition of the decoupling phenomenon using a fixing buffer, which stops the metabolic activity of the cells, demonstrates the role of the cellular metabolism in the impairment of the APC tandem fluorochromes.

However, the fixing of the cells causes the death of the cells and can especially cause the loss of functional epitopes.

Furthermore, the fixing of the cells is not one of the conditions when using the protocol for routine analysis of cells.

Accordingly, although the fixing of the cells makes it possible to inhibit the decoupling phenomenon, it is not a preferred method of the invention.

The inventors then studied, under similar leukocyte labeling conditions, the effect of the temperature by carrying out an incubation at 4° C. As a reminder, in routine assays, this incubation is performed at room temperature, that is to say between 18° C. and 25° C. inclusive.

The results are shown in FIGS. 22 and 24.

FIG. 22 represents the variation of the percentage of decoupling which is produced over time during the flow cytometry analysis of lymphocytes incubated at room temperature and of lymphocytes incubated at 4° C., of neutrophilic polynuclear cells incubated at room temperature and of neutrophilic polynuclear cells incubated at 4° C.

FIG. 24 represents the variation of the percentage of decoupling which appears during the flow cytometry analysis of monocytes labeled with an anti-CD45 antibody conjugated with the APC-Cy7 tandem fluorochrome, incubated at room temperature, and of monocytes incubated at 4° C.

As can be seen from FIGS. 22 and 24, at 4° C., all the percentage degradations of the APC-Cy7 tandem fluorochrome in the lymphocytes, the neutrophilic polynuclear leukocytes and the monocytes remain very low without any increase over time compared with the percentages observed on these same cells under the same conditions but with an incubation at room temperature.

The same results were obtained using the APC-H7 tandem fluorochrome.

These experiments confirm, once again, the importance of the cellular metabolism on the degradation of the APC tandem fluorochromes.

However, here again, incubation at 4° C. is not a condition of the protocol for routine analysis of cells. Furthermore, for incubation at 4° C., the duration of incubation is longer and requires more restrictive conditions. This method, although efficient, is not therefore a preferred method of the invention.

In order to block the metabolic activity of the cells, when the cells are maintained at room temperature, the inventors then tested the effect of sodium azide (NaN₃) which is often added to many conjugates.

It was then observed that 0.05% NaN₃ only weakly affected the decoupling phenomenon with lymphocytes but that 0.05% NaN₃ strongly inhibited the impairment of the APC-H7 tandem fluorochromes.

Similar effects were observed with monocytes and neutrophilic polynuclear leukocytes without affecting cell viability

This demonstrates that inhibiting metabolic activity leads to protection of the APC tandem fluorochromes.

However, sodium azide irreversibly inhibits cytochrome oxidase and is considered as being toxic for the technician and the environment.

The inventors then, and contrary to the prejudice existing in the art, studied the effect of exposure to light on the degradation of the APC tandem fluorochromes.

Indeed, according to the prior art, in general, and this being for all the fluorochromes, it is highly advisable to carry out the labeling of the cells in the absence of light and, for the APC tandem fluorochromes, and this point is particularly advanced in the manufacturer's instructions.

The inventors therefore challenged this prejudice and exposed immunolabeled blood cells to an artificial light source (neon tube) and compared the percentage of decoupling obtained under these conditions to the percentage of decoupling obtained when the immunolabeling was performed in the absence of light.

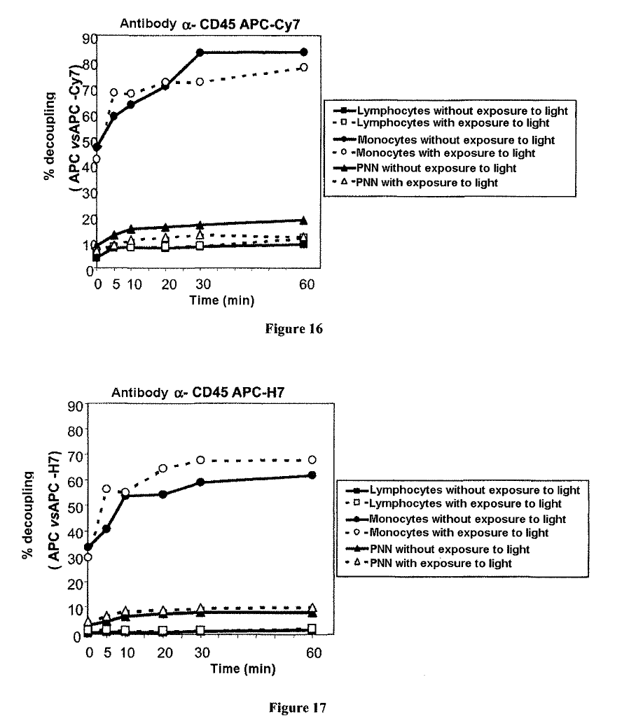

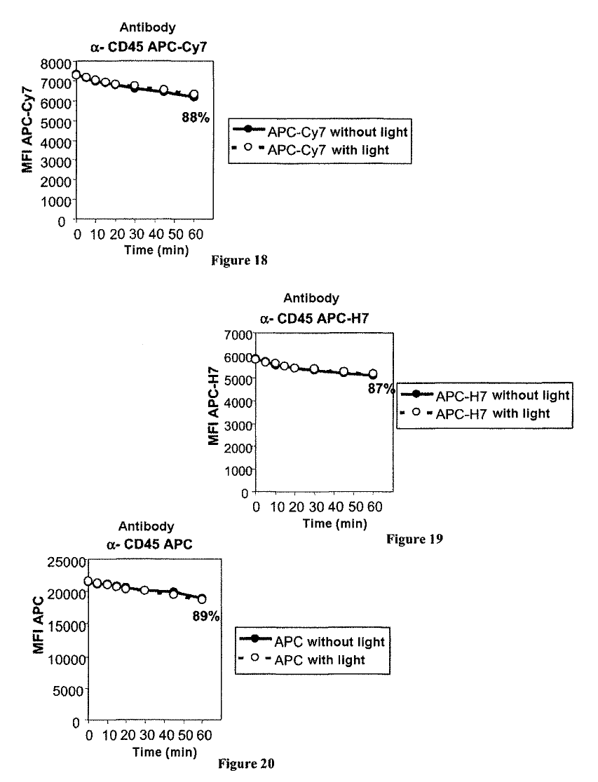

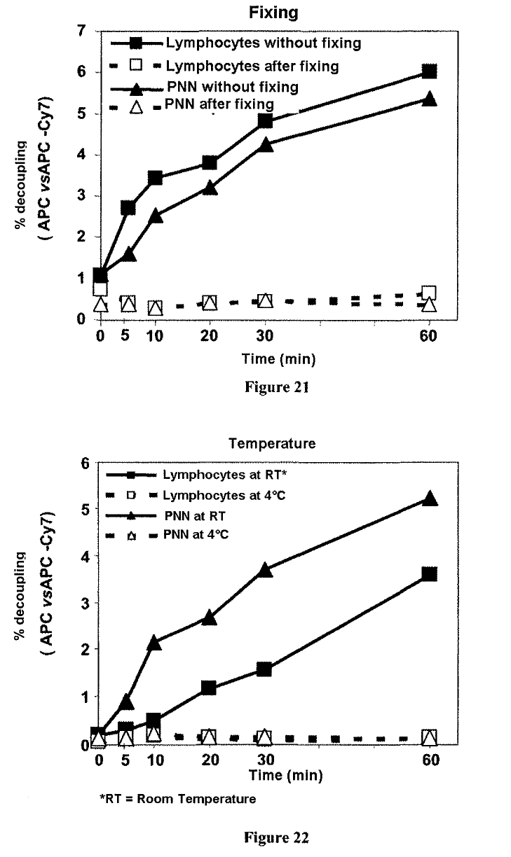

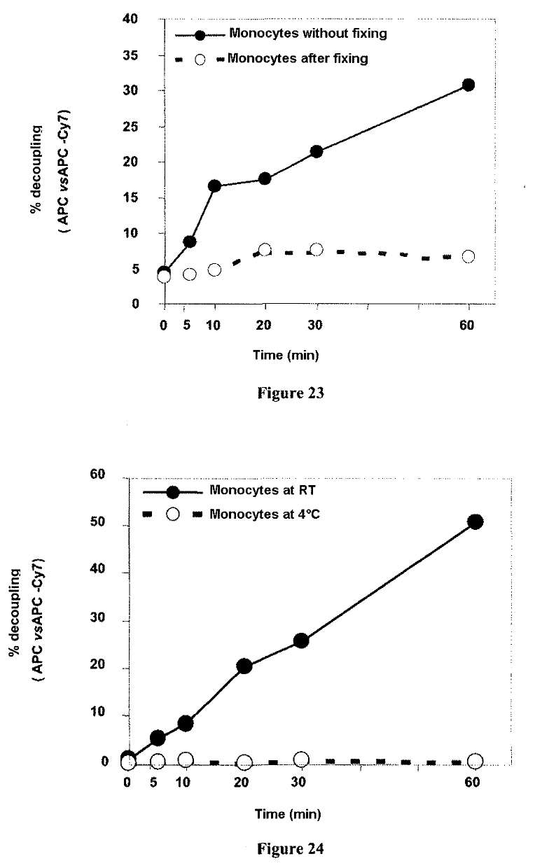

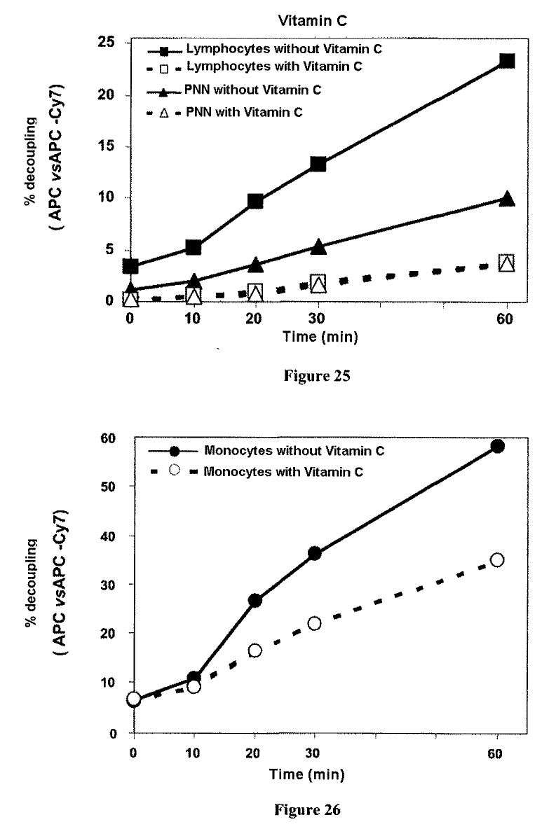

The results obtained are represented in FIGS. 16 to 20.

FIG. 16 represents the variation of the percentage of decoupling which is produced for lymphocytes, monocytes and neutrophilic polynuclear cells labeled with the anti-CD45 antibody conjugated with the APC-Cy7 tandem fluorochrome, as a function of time and during exposure to light or without exposure to light.

FIG. 17 represents the variation of the percentage of decoupling obtained on these same cells but labeled with the anti-CD45 antibody conjugated with the APC-H7 tandem fluorochrome, also in the presence of light or in the absence of light.

As can be seen from FIGS. 16 and 17, surprisingly, the percentage of degradation of the APC-Cy7 and APC-H7 tandem fluorochromes, for these three cell types was similar in the presence or in the absence of light over a short period of time of 1 h. This absence of an effect of light on the degradation of these tandems under these experimental conditions is accompanied by an equivalent bleaching of the APC fluorochromes and of the APC-Cy7 or APC-H7 tandem fluorochromes.

This hypothesis was confirmed by immunolabeling beads, that is to say in the absence of cells, with the anti-CD45 antibodies conjugated with the APC-Cy7 tandem fluorochrome, with the APC-H7 tandem fluorochrome and with the APC fluorochrome.

The variation in the mean fluorescence intensities (MFI) as a function of time is represented in FIGS. 18, 19 and 20.

More specifically, FIG. 18 represents the variation in the mean fluorescence intensity (MFI), as a function of time, of beads in the presence and in the absence of light, immunolabeled with anti-CD45 antibodies conjugated with the APC-Cy7 tandem.

FIG. 19 represents the variation in the mean fluorescence intensity (MFI), as a function of time, of beads labeled with anti-CD45 antibodies conjugated with the APC-H7 tandem, in the presence and in the absence of light.

FIG. 20 shows the variation in the mean fluorescence intensities (MFI), as a function of time, of beads labeled with anti-CD45 antibodies conjugated with the APC fluorochrome, in the presence and in the absence of light.

As can be seen from these figures, the mean fluorescence intensities of the three fluorochromes gradually decrease over time with similar kinetics in the absence or in the presence of light, suggesting that the absence of the effect of light is not linked to an underestimation of the bleaching of the APC fluorochrome.

Together, these experiments strongly exclude the hypothesis according to which exposure to light might play a role as catalyst for the degradation of the APC tandems under these experimental conditions.

The inventors thus overcame a prejudice in the art.

They then explored the potential role of an oxidation phenomenon.

The inventors then decided to study the possible influence of the presence of free radicals and/or of an underlying oxidation phenomenon in the degradation of the APC tandem fluorochromes.

Indeed, the results previously obtained show that the degradation of the APC tandem fluorochromes was produced in a higher proportion in the monocytes than in the lymphocytes and the neutrophilic polynuclear cells, which suggests that the decoupling phenomenon could depend on the properties of the type of cells studied.

One of the main functions of the monocytes and of the macrophages is phagocytosis and the killing of microorganisms, which depend on the production of superoxide and hydrogen peroxide (H₂O₂).

To test the influence of H₂O₂ on the degradation of the APC tandem fluorochromes, the inventors immunolabeled cells with an anti-CD45 antibody conjugated with APC-Cy7 and incubated them with or without H₂O₂.

The cells were analyzed for 20 to 60 minutes.

The results obtained show that 0.01 mM H₂O₂ does not modify the percentage of decoupling in the lymphocytes, while 0.1 mM H₂O₂ increased it.

A comparable role of hydrogen peroxide was observed with the monocytes and the neutrophilic polynuclear cells, without any effect on the viability of the cells, suggesting that the presence of free radicals and/or an oxidation phenomenon could play a role in the decoupling phenomenon.

It should be noted that, over time, hydrogen peroxide could induce the decoupling of the antibodies conjugated with the APC-Cy7 tandems attached to beads.

Because of the numerous disadvantages of the use of cell fixing, or of a low temperature or of sodium azide to inhibit the degradation of the APC tandem fluorochromes, the inventors tested the use of an antioxidant and/or free radical-scavenging agent on the decoupling phenomenon.

For that, cells immunolabeled with an anti-CD45 antibody conjugated with an APC tandem were incubated with or without vitamin C, used as an antioxidant and/or scavenging free radicals, although vitamin C is itself very fragile in solution and degraded by air, light and heat, before incubation at room temperature. The results are represented in FIGS. 25 and 26.

FIG. 25 represents the variation of the percentage of decoupling as a function of time which is produced in lymphocytes labeled with anti-CD45 antibodies conjugated with the APC-Cy7 tandem in the absence of vitamin C and in the presence of vitamin C at a final concentration of 1 millimolar (1 mM), and neutrophilic polynuclear cells labeled with anti-CD45 antibodies conjugated with the APC-Cy7 tandem in the absence of vitamin C and in the presence of vitamin C at a final concentration of 1 mM.

FIG. 26 represents the variation of the percentage of decoupling obtained on monocytes attached to the anti-CD45 antibody conjugated with the APC-Cy7 tandem in the absence of vitamin C and in the presence of vitamin C at a final concentration of 1 mM.

The percentage of degradation was monitored for 1 hour.

As can be seen from FIGS. 25 and 26, surprisingly, the presence of vitamin C strongly inhibits the percentage of decoupling over time in the lymphocytes, in the neutrophilic polynuclear leukocytes and, to a lesser degree, in the monocytes.

This result shows an important role of oxidation and/or of the presence of free radicals in the impairment of APC tandem conjugates on immunolabeled cells, which oxidation and/or presence of free radicals surprisingly is inhibited by vitamin C in solution.

This role was confirmed by subjecting beads Compbeads® to increasing concentrations of H₂O₂ (from 0 to 1 mM) with addition of 1 mM vitamin C. This addition of vitamin C blocks the impairment of the signal of the APC tandem at all the concentrations of H₂O₂ tested.

Accordingly, vitamin C is very advantageously used to prevent an abnormal signal in the APC channel.

Consequently, the invention is based on the discovery of the mechanism of impairment of the APC tandems on immunolabeled blood cells, that is to say a mechanism of oxidation and/or degradation by the free radicals of the APC tandem fluorochromes and not by a photodegradation phenomenon, as was thought to be the case in the prior art.

The invention is also based on the discovery that vitamin C effectively exerts its antioxidant and/or free radical-scavenging action even when it is in solution for a sufficient time to allow the analysis.

Although in the preceding text the invention has been described in relation to blood cells, it will clearly appear to a person skilled in the art that it is applicable to all types of cells that can be analyzed by flow cytometry, such as marrow cells or plant cells, and the like.

Likewise, although the use of vitamin C has made it possible to discover the mechanism giving rise to the phenomenon of decoupling, it will be clearly evident to a person skilled in the art that any antioxidant and/or free radical-scavenging agent that is nontoxic to the cells may be used in the invention.

In particular, ascorbic acid, in all its forms, is particularly suitable.

However, the use of ascorbic acid in its L form, that is to say vitamin C, has numerous advantages compared to other antioxidant and/or free radical-scavenging agents: it is soluble in aqueous medium and it is not toxic to cells at these concentrations.

Likewise, although the assays described and illustrated were performed with a final vitamin C concentration of 1 mM, assays were also performed at concentrations in the presence of vitamin C at a concentration of 0.1 millimolar (0.1 mM) and 10 millimolar (10 mM).

When the cells are lymphocytes, at a concentration of 0.1 mM, vitamin C already produces an effect, but only a reduced effect. Thus, lower concentrations of vitamin C are not suitable for obtaining a sufficient decrease of the percentage of decoupling. Furthermore, above 10 mM, the morphology and the viability of the lymphocytes are modified.

The best results are obtained at vitamin C concentrations of between 1 mM and 5 mM inclusive, with the lymphocytes.

Consequently, the preferred concentration, in the invention, of vitamin C, with lymphocytes, is 1 mM because it makes it possible to obtain optimum inhibition of the decoupling phenomenon with an optimum vitamin C concentration.

When the cells are monocytes, a sufficient decrease of the percentage of decoupling appears for a vitamin C concentration of 1 mM. This decrease exists up to a vitamin C concentration of 10 mM. Above this concentration of 10 mM, the viability and morphology of the monocytes are modified.

When the cells are neutrophilic polynuclear cells, a sufficient decrease of the percentage of decoupling is obtained with a vitamin C concentration of 0.25 mM.

This decrease exists up to a vitamin C concentration of 10 mM. Above 10 mM, the cells begin to undergo a decrease in their viability.

The best results are obtained with a vitamin C concentration of between 0.75 mM and 4 mM inclusive, for the neutrophilic polynuclear cells. Above 4 mM, the cells begin to undergo morphological impairment.

For this reason, the more preferred vitamin C concentration is between 0.75 mM and 2 mM inclusive, and the most preferred is 0.75 mM for the neutrophilic polynuclear cells

Furthermore, while in the preceding text the antioxidant and/or free radical-scavenging agent was added at the time of incubation in the labeling solution, it will clearly appear to persons skilled in the art that it may be added in advance, for example to the incubation buffer prepared in advance and used for labeling with the antibodies conjugated with the fluorochromes.

Consequently, the invention relates to the use of an antioxidant and/or free radical-scavenging agent, preferably ascorbic acid, and most preferably the L form of ascorbic acid, or vitamin C, in a method of analysis by flow cytometry.

Preferably, vitamin C is used at a final concentration in the labeling solution of between 0.1 mM and 10 mM inclusive, preferably 1 mM.

Likewise, the invention also relates to an incubation buffer used during the labeling of the cells which comprises an antioxidant and/or free radical-scavenging agent, preferably ascorbic acid, and most preferably vitamin C.

1. A method for analyzing cells by flow cytometry comprising a step for labeling the cells in a labeling solution comprising an incubation buffer and one or more labeling antibodies conjugated with at least one fluorochrome which is an APC tandem, wherein an antioxidant and/or free radical-scavenging agent is introduced into the labeling solution, in order to inhibit the degradation of said at least one fluorochrome.

2. The method as claimed in claim 1, wherein the cells are blood cells.

3. The method as claimed in claim 1, wherein the antioxidant and/or free radical-scavenging agent is ascorbic acid.

4. The method as claimed in claim 1, wherein the antioxidant and/or free radical-scavenging agent is vitamin C.

5. The method as claimed in claim 1, wherein the cells are lymphocytes and in that the antioxidant and/or free radical-scavenging agent is vitamin C present in the labeling solution at a final concentration of between 0.1 and 10 mM inclusive.

6. The method as claimed in claim 5, wherein the vitamin C is present in the labeling solution at a concentration of between 1 mM and 10 mM inclusive.

7. The method as claimed in claim 5, wherein the vitamin C is present in the labeling solution at a concentration of 1 mM.

8. The method as claimed in any claim 1, wherein the cells are monocytes and the antioxidant and/or free radical-scavenging agent is vitamin C present in the labeling solution at a final concentration of between 1 mM and 10 mM inclusive.

9. The method as claimed in claim 8, wherein the vitamin C is present in the labeling solution at a concentration of 1 mM.

10. The method as claimed in claim 1, wherein the cells are neutrophilic polynuclear cells and the antioxidant and/or free radical-scavenging agent is vitamin C present in the labeling solution at a final concentration of between 0.25 mM and 10 mM inclusive

11. The method as claimed in claim 10, wherein the vitamin C is present in the labeling solution at a concentration of between 0.75 mM and 4 mM inclusive.

12. The method as claimed in claim 10, wherein the vitamin C is present in the labeling solution at a concentration of between 0.75 mM and 2 nM inclusive.

13. The method as claimed in claim 10, wherein the vitamin C is present in the labeling solution at a concentration of 0.75 mM.

14. An incubation buffer, comprising an antioxidant and/or free radical-scavenging agent.

15. The buffer as claimed in claim 14, wherein the antioxidant and/or free radical-scavenging agent is ascorbic acid.

16. The buffer as claimed in claim 14, wherein the antioxidant and/or free radical-scavenging agent is vitamin C.
